(19) United States
(12) Patent Application Publication
(10) Pub. No.: US 2009/0221652 A1
(43) Pub. Date: Sep. 3, 2009

(54) COMBINATIONS OF METFORMIN AND MEGLITINIDE

(76) Inventor: Bard J. Geesaman, Seattle, WA (US)

Correspondence Address: LANDO & ANASTASI, LLP;E2023, One Main Street, Suite 1100, Cambridge, MA, 02142, US

(21) Appl. No.: 12/092,795

(22) PCT Filed: Nov. 7, 2006
(86) PCT No.: PCT/US06/43376

§ 371 Date: Mar. 2, 2009

Related U.S. Application Data
(60) Provisional application No. 60/734,528, filed on Nov. 7, 2005.
(60) Provisional application No. 60/753,754, filed on Dec. 23, 2005.
(60) Provisional application No. 60/773,087, filed on Feb. 14, 2006.

(51) Int. Cl. A61K 31/403; A61K 31/4418; A61K 31/197; A61K 31/155; A61P 3/10

(52) U.S. Cl. 514/357; 514/412; 514/563; 514/635

(57) ABSTRACT

Methods of treating glucose related disorders, for example by administering a meglitinide such as mitiglinide, repaglinide, or nateglinide, optionally in combination with a biguanide such as metformin, are described herein.

### CROSS-REFERENCE TO RELATED APPLICATIONS

This application claims priority to U.S. Application Ser. No. 60/734,528, filed on Nov. 7, 2005, U.S. Application Ser. No. 60/753,754, filed Dec. 23, 2005, and U.S. Application Ser. No. 60/773,087, filed Feb. 14, 2006, the entire contents of each of which are incorporated herein by reference.

### FIELD OF THE INVENTION

The invention relates to glucose related disorders.

### SUMMARY

In one aspect, the invention features a method of treating, or prophylactically treating, a subject, preferably a subject at risk for having, or having, type II diabetes, or another condition described herein. (Treatment, as used herein, means reducing a symptom or unwanted characteristic of the condition or disorder. Prophylactically, as used herein, means preventing a disorder, e.g., delaying its onset, in a subject). The method includes: administering to a subject a meglitinide, e.g., mitiglinide, repaglinide, or nateglinide, preferably mitiglinide, at least twice a day (and preferably no more than twice a day) wherein at least one meal is consumed by the subject without the administration of a dose of a meglitinide, and preferably administering, preferably orally, a biguanide, e.g., metformin, at least one time per day (e.g., one time a per day or two times per day); thereby treating said subject.

In a preferred embodiment, the method includes: administering, preferably orally, a meglitinide, e.g., mitiglinide, repaglinide, or nateglinide, preferably mitiglinide, with at least one meal but not every meal in a day. For example, a first dose is administered with a first meal, and a second does is administered independent of a meal, e.g., before bedtime.

In a preferred embodiment, the method includes: administering a first dose with a first meal; administering a second dose with a second meal; and wherein a third meal is consumed by the subject without a dose of a meglitinide (e.g., mitiglinide, repaglinide, or nateglinide, preferably mitiglinide) wherein the terms first, second, and third are used merely to identify the meal and dose and do not imply an order of meal or dose. For example, the first meal can be the first of the day, the second meal can be the last of the day, and the third meal can be the midday meal.

In a preferred embodiment, the method includes administering a dose with breakfast, or the first meal of the day; administering a dose with the last, or evening meal, and wherein the subject also consumes a third meal or midday meal.

In a preferred embodiment, the method includes administering in combination a dose of a meglitinide (e.g., mitiglinide, repaglinide, or nateglinide, preferably mitiglinide) and a dose of a biguanide (e.g., metformin) with breakfast, or the first meal of the day; administering in combination a dose of a meglitinide (e.g., mitiglinide, repaglinide, or nateglinide, preferably mitiglinide) and a dose of a biguanide (e.g., metformin) with the last, or evening meal, and wherein the subject also consumes a third meal or midday meal and does not take a dose of a meglitinide with the third or midday meal.

In a preferred embodiment, the method includes administering in combination a dose of mitiglinide and a dose of metformin with breakfast, or the first meal of the day; administering in combination a dose of mitiglinide and a dose of metformin with the last, or evening meal, and wherein the subject also consumes a third meal or midday meal and does not take a dose of a meglitinide with the third or midday meal.

In a preferred embodiment, the method includes administering in combination a dose of repaglinide and a dose of metformin with breakfast, or the first meal of the day; administering in combination a dose of a repaglinide and a dose of metformin with the last, or evening meal, and wherein the subject also consumes a third meal or midday meal and does not take a dose of a meglitinide with the third or midday meal.

In a preferred embodiment no more than two meglitinide (e.g., mitiglinide, repaglinide, or nateglinide, preferably mitiglinide) administrations per day are administered to the subject.

In a preferred embodiment no more than one meglitinide (e.g., mitiglinide, repaglinide, or nateglinide, preferably mitiglinide) administration per day is administered to the subject.

In a preferred embodiment no more than two biguanide (preferably metformin) administrations per day are administered to the subject.

In a preferred embodiment no more than one biguanide (preferably metformin) administration per day is administered to the subject.

In a preferred embodiment two, or no more than two, meglitinide, e.g., mitiglinide, repaglinide, or nateglinide, administrations per day and two, or no more than two, biguanide administrations per day are administered to the subject. In a particularly preferred embodiment the meglitinide is mitiglinide and the biguanide is metformin.

In a preferred embodiment one and only one meglitinide, e.g., mitiglinide, repaglinide, or nateglinide administration per day and one and only one biguanide administration per day is administered to the subject. In a particularly preferred embodiment the meglitinide is mitiglinide and the biguanide is metformin.

In a preferred embodiment at least one meglitinide e.g., mitiglinide, repaglinide, or nateglinide, administration is administered with a metformin administration, preferably as a combined unit dosage formulation. For example a mitiglinide, administration is administered with a metformin administration, preferably as a combined unit dosage formulation.

In a preferred embodiment, metformin and a meglitinide such as mitiglinide, repaglinide, or nateglinide are formulated into a single unit dosage such that at least a portion of the two compounds are combined as a salt (e.g., the meglitinide being the acidic component and metformin being the basic component). Exemplary salts include metformin:mitiglinide, metformin:repaglnide; and metformin:nateglinide.

In another preferred embodiment, where the two agents are combined into a single unit dosage, at least a portion of the metformin is in the form of an HCl salt. In some preferred embodiments, at least a portion of the meglitinide is in the form of a commercially available salt, for example a calcium salt of mitiglinide.

In a preferred embodiment each meglitinide, e.g., mitiglinide, repaglinide, or nateglinide, administration is administered with a metformin administration, preferably as a combined unit dosage formulation. For example, a mitiglinide, administration is administered with a metformin administration, preferably as a combined unit dosage formulation. In a preferred embodiment, metformin and a meglitinide such as mitiglinide, repaglinide, or nateglinide are formulated into a single unit dosage such that at least a portion of the two compounds are combined as a salt (e.g., the meglitinide being the acidic component and metformin being the basic component).

In a preferred embodiment a combined administration comprises administering a first dosage formulation comprising metformin and a second dosage formulation comprising a meglitinide, e.g., mitiglinide, repaglinide, or nateglinide. For example a combined administration comprises administering a first dosage formulation comprising metformin and a second dosage formulation comprising mitiglinide.

In a preferred embodiment the combined administration comprises administering a combined unit dosage formulation comprising both metformin and a meglitinide, e.g., mitiglinide, repaglinide, or nateglinide. For example, the combined administration comprises administering a combined unit dosage formulation comprising both metformin and a meglitinide such as mitiglinide, repaglinide or nateglinide. For example, the combined unit dosage formulation can include a salt of metformin and a meglitinide (e.g., metformin:mitiglinide, metformin:repaglnide; and metformin:nateglinide). In some embodiments the combined unit dosages include a commercially available salt of one or more of the agents.

In a preferred embodiment two, or no more than two, combined unit dosage formulations (including both a meglitinide, e.g., mitiglinide, repaglinide, or nateglinide and a biguanide) are administered per day to the subject. In a particularly preferred embodiment the meglitinide is mitiglinide and the biguanide is metformin.

In a preferred embodiment one, and only one, combined unit dosage formulation (including both a meglitinide, e.g., mitiglinide, repaglinide, or nateglinide and a biguanide) are administered per day to the subject. In a particularly preferred embodiment the meglitinide is mitiglinide and the biguanide is metformin.

In preferred embodiments an additional dosage, e.g. one which is not a combined unit dosage formulation is administered per/day.

In a preferred embodiment the combined administration comprises administering a dosage formulation comprising both metformin and a meglitinide, e.g., mitiglinide, repaglinide, or nateglinide, only once per day. For example, the combined administration comprises administering a dosage formulation comprising both metformin and mitiglinide only once per day. For example, the combined unit dosage formulation can include a salt of metformin and a meglitinide.

In a preferred embodiment the combined administration comprises administering a combined unit dosage formulation comprising both metformin and a meglitinide, e.g., mitiglinide, repaglinide, or nateglinide, only twice per day. For example, the combined administration comprises administering a combined unit dosage formulation comprising both metformin and mitiglinide only twice per day. For example, the combined unit dosage formulation can include a salt of metformin and a meglitinide. In this, or other embodiments where two or three dosages are given per/day, they are preferably given with 16, 12, or 8 hours of one another.

In a preferred embodiment at least one and preferably two meglitinide administrations, e.g., mitiglinide, repaglinide, or nateglinide, preferably mitiglinide administrations are provided within 30 minutes of a meal.

In a preferred embodiment at least one and preferably two meglitinide administrations, e.g., mitiglinide, repaglinide, or nateglinide, preferably mitiglinide administrations are provided within 30 minutes before a meal.

In a preferred embodiment a meglitinide, e.g., mitiglinide, repaglinide, or nateglinide, preferably mitiglinide administration, preferably a combined meglitinide and metformin administration, is provided prior to each of two meals of the day that meet a predetermined characteristic, for example, they contain a predetermined sugar/carbohydrate content, preferably the two meals with the greatest sugar/carbohydrate content.

In a preferred embodiment a meglitinide administration (e.g., mitiglinide, repaglinide, or nateglinide, preferably mitiglinide, administration) preferably a combined meglitinide and metformin administration, is provided prior to each of one or two meals in a day. In a preferred embodiment it is prior to one or both of the largest two meals of the day, for example the two meals having the highest calorie content.

In a preferred embodiment a meglitinide administration (e.g., mitiglinide, repaglinide, or nateglinide, preferably mitiglinide, administration) preferably a combined meglitinide and metformin administration, is provided prior to each of one or two meals in a day, wherein one or both of the meals, independently, is at least 500, 1,000, or 1,500 calories. In a preferred embodiment at least 500, 1,000, or 1,500 calories of the meal comes from carbohydrates.

In a preferred embodiment, at least one and preferably both meglitinide, e.g., mitiglinide, repaglinide, or nateglinide, preferably mitiglinide administrations are provided during a meal.

In a preferred embodiment the method further comprises instructing the subject (e.g., by providing written or oral instructions to the subject or to a caregiver who will transmit instructions the subject) to take a composition described herein, e.g., in a regimen described herein. For example, the method can include instructing the subject to take a meglitinide (e.g., mitiglinide, repaglinide, or nateglinide) according to a regime disclosed herein, e.g., no more than one or two times per day (for example, even in instances where the subject will eat three or more meals in the day). For example, the method further comprises instructing the subject to take mitiglinide according to a regime disclosed herein, e.g., no more than one or two times per day.

In a preferred embodiment the method further comprises instructing the subject to take a meglitinide (e.g., mitiglinide, repaglinide, or nateglinide) two times per day but that one or both of the administrations do not need to occur just prior to or with a meal. For example, the method further comprises instructing the subject to take mitiglinide two times per day but that one or both of the administrations do not need to occur just prior to or with a meal.

In a preferred embodiment the unit dose of mitiglinide is less than or equal to 5, 4, 3, 2, or 1 mg.

In a preferred embodiment the unit dose of repaglinide is less than or equal to 5, 4, 3, 2, 1, 0.5 or 0.25 mg.

In a preferred embodiment the unit dose of nateglinide is less than or equal to 120, 110, 100, 90, 80, 70, 60, 50, 40, 30, or 20 mg.

In a preferred embodiment the unit dose of the mitiglinide is from about 5 mg to about 40 mg.

In a preferred embodiment the unit dose of the repaglinide is from about 0.25 mg to about 12 mg.

In a preferred embodiment the unit dose of the nateglinide is from about 30 mg to about 300 mg.

In a preferred embodiment the unit dose (or daily dose) of the meglitinide, (e.g., mitiglinide, repaglinide, or nateglinide) is reduced by at least 10, 20, 50, or 75% as compared with what would be given to the subject in meglitinide monotherapy.

In some embodiments the subject is: drug naïve.

In other embodiments, the subject is not drug naïve, e.g., the subject has been treated with one or more of a biguanide, preferably metformin, a thiazolidinedione (e.g., a TZD such as rosiglitazone or pioglitazone), an alpha glucosidase inhibitor, such as acarbose or voglibose, a glucagon-like peptide agonist (e.g., a GLP-1 agonist), or a dipeptidylpeptidase 4 (e.g., DPP4) inhibitor within 10, 30, 60, or 180 days of administering the treatment.

In some embodiments the method includes evaluating a drug in a naïve subject, or a subject who is not drug naïve, for treatment with method described herein. For example, the method can include evaluating a subject previously or currently treated with a biguanide, preferably metformin, for treatment with a method described herein.

In a preferred embodiment the method includes administering less than about 800 mg of metformin per day, for example, from about 350 mg to about 750 mg per day of metformin.

In a preferred embodiment the method includes administering at least about 800 mg of metformin per day.

In a preferred embodiment the method includes administering from about 1000 mg to about 2000 mg of metformin per day, e.g., from about 1200 mg to about 1600 mg per day.

In a preferred embodiment, the method includes titrating a subject. For example, the method includes titrating a subject for a meglitinide (e.g., mitiglinide, repaglinide, or nateglinide). In a preferred embodiment, the subject is administered increasing doses of the meglitinide, and evaluating the subject for tolerance to the meglitinide as the dose is increased. In embodiments where the subject is titrated for mitiglinide, exemplary doses include 1, 2.5, 5, 7.5, 10, 12.5, 15, 17.5, 20, 22.5, 25, 27.5, 30, 32.5, 35, 37.5, and 40 mgs. In some embodiments the patient is being administered a constant dose of a biguanide such as metformin while being titrated with the meglitinide (e.g., where the patient has already been on a treatment regime that includes a biguanide such as metformin). In some embodiments, the patient is being titrated with a biguanide such as metformin while being titrated for the meglitinide. Exemplary doses of metformin include 250, 500, 750, 1000, 1500, and 2000 mgs.

In another aspect, the method includes titrating a subject for a meglitinide such as mitiglinide, repaglinide, or nateglinide, e.g., where the subject is not naïve to a meglitinide, e.g., the patient is changing from one meglitinide to another meglitinide. For example, the method includes titrating a subject for a new meglitinide (e.g., mitiglinide, repaglinide, or nateglinide) to the patient. In a preferred embodiment, the subject is administered increasing doses of the new meglitinide, and evaluating the subject for tolerance to the new meglitinide as the dose is increased. In some embodiments where the subject is titrated for a new meglitinide (i.e., where the patient is not naïve to meglitinides), titration of the new meglitinide can begin at a relatively higher does, e.g., where the new meglitinide is mitiglinide, the relatively higher dose can begin at 10, 12.5, 15, 17.5, 20, 22.5, 25, 27.5, 30, 32.5, 35, 37.5, and 40 mgs of the mitiglinide. In some embodiments the patient is being administered a constant dose of a biguanide such as metformin while being titrated with the meglitinide. In some embodiments, the patient is being titrated with a biguanide such as metformin while being titrated for the meglitinide. Exemplary doses of metformin include 250, 500, 750, 1000, 1500, and 2000 mgs.

In another aspect, the invention includes instructing a patient to self administer a meglitinide such as mitiglinide, repaglinide, or nateglinide and optionally a biguanide according a method described herein.

In another aspect, the invention features a method of treating prediabetes, lowering blood glucose in a subject, decreasing hemoglobinA1c in a subject, inhibiting gluconeogenesis in a subject, decreasing post-prandial glucose in a subject (e.g., treating post-prandial hyperglycemia), treating type II diabetes, or treating a metabolic disorder, comprising administering a treatment described herein, for example, administering, orally, a meglitinide, e.g., mitiglinide, repaglinide, or nateglinide, preferably mitiglinide, not more than two times per day; administering, orally, and a biguanide, e.g., metformin, at least one time per day, thereby lowering blood glucose in said subject.

In another aspect, the invention features a method to treat pre-diabetes or type 2 diabetes with various combinations of mitiglinide and metformin, starting with sub effective doses of each and elevating doses to combinations of known effective doses of each, so as to affect a true pharmacological synergy of the two drug classes. The combination can be administered to pancreatic β-cells in culture to determine the effects of the combination on glucose-stimulated insulin release; combinations can also be administered to cells to determine their effect on β-cell viability (inhibition of apoptosis). In another aspect, variable combinations of mitiglinide and metformin can be administered to rodents, dogs, monkeys or humans to determine their effect on β-cell functions (c-peptide, HbA1c) in addition to β-cell survival in type 2 diabetes. Rodent models of Type 2 diabetes may be diet-induced obesity, ob/ob, db/db mice; or Zucker fatty rats. Alternative models include diet-induced obesity in dogs or monkeys). Exemplary doses administered to humans include: 100, 150, 200, 250, 300, 350, 500, 750, 1500, 2000 or 3000 mg of metformin in combination with any of the following doses of mitiglinide: 5, 10, 15, 20, 25, 30, 35, 40, 50 or 60 mg.

In another aspect, the invention features a method of treating, preventing, or reducing the risk of a myocardial infarction comprising administering to a subject an agent described herein, e.g., a meglitinide, e.g., mitiglinide, repaglinide, or nateglinide, preferably mitiglinide. The meglitinide can be administered alone or in combination. In a preferred embodiment, the method includes administering a combination of metformin and a meglitinide, e.g., mitiglinide, repaglinide, or nateglinide, preferably mitiglinide to a subject who has had a myocardial infarction, thereby reducing the risk of an additional myocardial infarction in the subject. The subject can have a disorder described herein, for example, metabolic syndrome, prediabetes, or type 2 diabetes.

In another aspect, the invention includes a pharmaceutical composition disclosed herein, e.g., a pharmaceutical composition comprising a meglitinide, (e.g., mitiglinide, repaglinide, or nateglinide), preferably mitiglinide, and a biguanide e.g., metformin, in amounts sufficient to allow treatment of a subject. In some embodiments, the pharmaceutical composition is formulated to provide once daily dosing, for example, by providing extended release of one or more of the agents described herein.

In a preferred embodiment the unit dose of mitiglinide is from about 5 mg to about 40 mg.

In a preferred embodiment the unit dose of repaglinide is from about 0.25 mg to about 12 mg.

In a preferred embodiment the unit dose of nateglinide is from about 30 mg to about 300 mg.

In a preferred embodiment the composition includes from about 350 mg to about 1500 mg of a biguanide.

In a preferred embodiment the biguanide is metformin.

In a preferred embodiment the composition includes an additional therapeutic agent, for example an anti-diabetic agent. Examples of anti-diabetic agents include a glucosidase inhibitor, a thiazolidinedione (e.g., TZD), an insulin sensitizer, a glucagon-like peptide-1 (GLP-1), insulin, a PPAR α/γ dual agonist, an aP2 inhibitor and/or a DPP4 inhibitor. Examples of a glucosidase inhibitor include acarbose (disclosed in U.S. Pat. No. 4,904,769), voglibose, miglitol (disclosed in U.S. Pat. No. 4,639,436), which may be administered in a separate dosage form or the same dosage form. Examples of a PPARγ agonist includes a thiazolidinedione (e.g., TZD) such as rosiglitazone (AVANDIA®), pioglitazone (ACTOS®), englitazone, and darglitazone, which may be administered in a separate dosage form or the same dosage form. An example of a DPP4 inhibitor includes PHX1149, which is being developed by Phenomix®.

The compositions described herein can also be administered with an anti-hyperglycemic agent such as insulin or with glucagon-like peptide-1 agonist (GLP-1) or analogs thereof, such as GLP-1(1-36) amide, GLP-1(7-36) amide, GLP-1(7-37) (as disclosed in U.S. Pat. No. 5,614,492, the disclosure of which is incorporated herein by reference), which may be administered via injection, or by transdermal or buccal devices. Treatments can include incretin mimetics such as exenatide (BYETTA®) and DPP-IV inhibitors such as sitagliptin (JANUVIA) and vildagliptin (GALVUS).

The compositions described herein can also be administered with an antihypertensive, such as an angiotensin II blocker (ARB) (e.g., irbesartan (AVAPRO), candesartan (ATACAND), losartan (COZAAR), or valsartan (DIOVAN)), or an angiotensin converting enzyme (ACE) inhibitor such as captopril (Capoten), benazepril (Lotensin), enalapril (Vasotec), lisinopril_(Prinivil, Zestril) fosinopril (Monopril), ramipril (Altace), perindopril (Aceon), quinapril (Accupril), moexipril (Univasc), and trandolapril (Mavik).

The details of one or more embodiments of the invention are set forth in the description below.

### DETAILED DESCRIPTION

Methods of treating, prophylactically treating, and preventing type II diabetes, prediabetes, and related metabolic disorders are described. Also described are compositions and dosages for treating and preventing such disorders.

A biguanide is an agent that can be used in the treatment or prevention of type II diabetes and/or improvement of glycemic control. While not being bound by a mechanism of action, it is thought that the biguanides as a class have a two-fold mechanism of action. They enhance peripheral muscle glucose uptake and utilization and inhibit glucose release from the live, thereby decreasing insulin resistance. A commonly used biguanide is metformin. Other biguanides include phenformin and buformin.

Meglitinides are another class of agents used in the treatment or prevention of type II diabetes and/or improvement of glycemic control. While not being bound by a mechanism of action, it is thought that the meglitinides promote the secretion of insulin by inhibiting the APT-sensitive K⁺ channel current through bonding with pancreatic beta cells' sulfonylurea receptors. Examples of meglitinides include repaglinide, nateglinide, and mitiglinide.

In some instances it is desirable to treat a subject with a combination of agents, for example a combination of a biguanide and a meglitinide, preferably a combination of metformin and a meglitinide, for example mitiglinide.

As used herein, “administered in combination” or a combined administration of two agents means that two or more agents are administered to a subject at the same time or within an interval such that there is overlap of an effect of each agent on the patient. Preferably they are administered within 15, 10, 5, or 1 minute of one another. Preferably the administrations of the agents are spaced sufficiently close together such that a combinatorial effect is achieved. The agents can be administered simultaneously, for example in a combined unit dose (providing simultaneous delivery of both agents). Alternatively, the agents can be administered at a specified time interval, for example, an interval of minutes, hours, days or weeks. Generally, the agents are concurrently bioavailable, e.g., detectable, in the subject.

In a preferred embodiment, the agents are administered essentially simultaneously, for example two unit dosages administered at the same time, or a combined unit dosage of the two agents. In another preferred embodiment, the agents are delivered in separate unit dosages. The agents can be administered in any order, or as one or more preparations that includes two or more agents. In a preferred embodiment, at least one administration of one of the agents, e.g., the first agent, is made within minutes, one, two, three, or four hours, or even within one or two days of the other agent, e.g., the second agent. In some cases, combinations can achieve synergistic results, e.g., greater than additive results, e.g., at least 20, 50, 70, or 100% greater than additive.

Unit Dosage Formulations and Administration

Compounds and compositions disclosed herein can be used to treat or prevent type II diabetes, prediabetes, or other conditions described herein.

As discussed elsewhere herein, subjects are treated with a combination of a biguanide, preferably metformin, and a meglitinide, e.g., mitiglinide, repaglinide, or nateglinide, preferably mitiglinide. These agents are generally administered orally. They can be administered separately, e.g., in separate unit dosage formulations, e.g., one pill or capsule containing metformin and a separate pill or capsule containing a meglitinide. It is preferred, however that the two agents are provided as a combined formulation. A combined formulation is one in which the two agents provided together in a unit dosage formulations, e.g., one pill or capsule containing metformin and a meglitinide.

In another embodiment, the first and second agent (preferably metformin and a meglitinide) are further combined with a third agent, and can be, for example, co-formulated as a single pill (e.g., tablet, gel, or other dosage unit) for administration together. For example, the administration of the dose formula can be one, two or three times daily, e.g., as required depending on the number and volume of meals. For example, the dose formula can be two times per day in instances where a subject has eaten at least three meals in the day.

In some embodiments, one or more of the agents is formulated as a pharmaceutically acceptable salt. Examples of pharmaceutically acceptable include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undecanoate. Salts derived from appropriate bases include alkali metal (e.g., sodium), alkaline earth metal (e.g., magnesium, calcium), ammonium and N-(alkyl)₄⁺ salts. This invention also envisions the quaternization of any basic nitrogen-containing groups of the compounds disclosed herein. Water or oil-soluble or dispersible products may be obtained by such quaternization.

In some embodiments, for example, where metformin and a meglitinide (e.g., mitiglinide, repaglinide, or nateglinide), preferably mitiglinide are formulated into a single unit dosage, the two compounds can be combined as a salt. Mitiglinide has as its active component a cis-hexahydroindoline carboxamide bearing a free carboxylic acid functionality, and metformin is a N,N-dimethyl biguanide, which is a basic functionality. As marketed as individual drugs, mitiglinide (GLUFAST®) is present as its calcium salt, and metformin (GLUCOPHAGE®) is its hydrochloride salt. Mitiglinide free acid and metformin free base are capable of forming a salt when combined, as shown in Scheme 1, below.

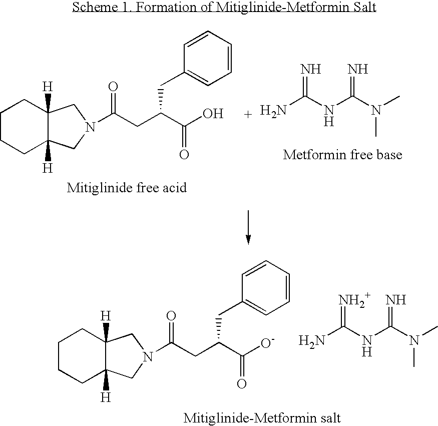

The salt formation is a simple acid-base reaction, consisting of a proton transfer of mitiglinide free acid to the basic biguanide function of metformin free base, in an appropriate solvent. Mitiglinide and metformin are both chemically stable molecules, and the only reactive functionalities are the acid and base functional groups, respectively. The resulting salt should therefore be a chemically stable entity, based on the known stability of each component.

While the free base of the two agents (e.g., mitiglinide and metformin) are shown above, in some embodiments, commercially available salts (e.g., the calcium salt of mitiglinide and the hydrochloride salt of metformin) are solubilized in an appropriate solvent and converted to a mitiglinide:metformin salt. For example, the calcium component of the mitiglinide can combine with the chloride component of the metformin to provide a calcium chloride salt, which can be discarded from the new mitiglinide:metformin salt.

In a manner similar to that described above for the formation of a mitiglinide:metformin salt, other meglitinides can also form meglitinide:metformin salts having the carboxylic acidic component of the meglitinide acting as described above with mitiglinide. Exemplary salts include repaglinide:metformin and nateglinide:metformin.

In embodiments where the metformin and meglitinide are not administered in stoichiometric equivalent amounts, the agent administered in excess is generally present in another form (e.g., is not a combined salt form with the other agent). For example, where metformin is administered in stoichiometric excess, the excess amount of metformin can be present as the hydrochloride salt.

Unit dosage formulations for metformin and a meglitinide, e.g., mitiglinide, repaglinide, or nateglinide include those outlined below. Amounts are given in milligrams.

In some applications, the composition will be administered once per day to achieve a daily metformin dose of 500-3000 mg and a daily meglitinide dose having a desired effect e.g., a daily mitiglinide dose of 15-300 mg, for example in instances where at least two meals a day are consumed by the subject, for example, where at least one meal is consumed by the subject without the administration of a dose of a meglitinide such as mitiglinide, repaglinide, or nateglinide. The following unit dosage forms, while not limiting for this use, can be used for such treatment regime. The exact amounts given below are illustrative. The amount of an agent can vary as long as the therapeutic goal is achieved, e.g., the amount of an agent can vary by up to 10, 20, 25, or 50%. As would be understood by one of skill in the art, the metformin would be administered with only one of the meglitinide agents in the table below. However, dosage rages of three exemplary meglitinide agents are provided.

| Metformin | Mitiglinide | Repaglinide | Nateglinide |
|---|---|---|---|
| 250-750 | 5-300 | 0.5-100 | 60-600 |
| 500 | 15 | 1 | 60 |
| 500 | 60 | 2 | 90 |
| 500 | 80 | 6 | 120 |
| 500 | 120 | 10 | 240 |
| 500 | 200 | 25 | 360 |
| 500 | 300 | 100 | 600 |
| 750-1500 | 5-300 | 0.5-100 | 60-600 |
| 1000 | 15 | 1 | 60 |
| 1000 | 60 | 2 | 90 |
| 1000 | 80 | 6 | 120 |
| 1000 | 120 | 10 | 240 |
| 1000 | 200 | 25 | 360 |
| 1000 | 300 | 100 | 600 |
| 2000-3500 | 5-300 | 0.5-100 | 60-600 |
| 3000 | 15 | 1 | 60 |
| 3000 | 60 | 2 | 90 |
| 3000 | 80 | 6 | 120 |
| 3000 | 120 | 10 | 240 |
| 3000 | 200 | 25 | 360 |
| 3000 | 300 | 100 | 600 |

In some applications, the composition will be administered twice per day to achieve a daily metformin dose of 500-3000 mg and a daily meglitinide dose having a desired effect e.g., a daily mitiglinide dose of 15-300 mg, for example in instances where at least three meals a day are consumed by the subject, for example, where at least one meal is consumed by the subject without the administration of a dose of a meglitinide such as mitiglinide, repaglinide, or nateglinide. The following unit dosage forms, while not limited for this use, can be used for such treatment regime. The exact amounts given below are illustrative. The amount of an agent can vary as long as the therapeutic goal is achieved, e.g., the amount of an agent can vary by up to 10, 20, 25, or 50%. As would be understood by one of skill in the art, the metformin would be administered with only one of the meglitinide agents in the table below. However, dosage rages of three exemplary meglitinide agents are provided.

| Metformin | Mitiglinide | Repaglinide | Nateglinide |
|---|---|---|---|
| 125-375 | 5-200 | 0.5-75 | 60-400 |
| 250 | 7.5 | 1 | 60 |
| 250 | 30 | 2 | 90 |
| 250 | 40 | 4 | 120 |
| 250 | 60 | 10 | 150 |
| 250 | 100 | 25 | 200 |
| 250 | 150 | 50 | 300 |
| 375-750 | 5-200 | 0.5-75 | 60-400 |
| 500 | 7.5 | 1 | 60 |
| 500 | 30 | 2 | 90 |
| 500 | 40 | 4 | 120 |
| 500 | 60 | 10 | 150 |
| 500 | 100 | 25 | 200 |
| 500 | 150 | 50 | 300 |
| 1000-2000 | 5-200 | 0.5-75 | 60-400 |
| 1500 | 7.5 | 1 | 60 |
| 1500 | 30 | 2 | 90 |
| 1500 | 40 | 4 | 120 |
| 1500 | 60 | 10 | 150 |
| 1500 | 100 | 25 | 200 |
| 1500 | 150 | 50 | 300 |

In some applications, the composition will be administered three times per day to achieve a daily metformin dose of 500-3000 mg and a daily meglitinide dose having a desired effect e.g., a daily mitiglinide dose of 15-300 mg, for example in instances where at least four meals a day are consumed by the subject, for example, where at least one meal is consumed by the subject without the administration of a dose of a meglitinide such as mitiglinide, repaglinide, or nateglinide. The following unit dosage forms, while not limited for this use, can be used for such treatment regime. The exact amounts given below are illustrative. The amount of an agent can vary as long as the therapeutic goal is achieved, e.g., the amount of an agent can vary by up to 10, 20, 25, or 50%. As would be understood by one of skill in the art, the metformin would be administered with only one of the meglitinide agents in the table below. However, dosage rages of three exemplary meglitinide agents are provided.

| Metformin | Mitiglinide | Repaglinide | Nateglinide |
|---|---|---|---|
| 125-250 | 5-150 | 0.5-50 | 60-300 |
| 166 | 5 | 1 | 60 |
| 166 | 20 | 2 | 90 |
| 166 | 26.6 | 4 | 120 |
| 166 | 40 | 10 | 150 |
| 166 | 66.6 | 20 | 180 |
| 166 | 100 | 33 | 200 |
| 250-400 | 5-150 | 0.5-50 | 60-300 |
| 333 | 5 | 1 | 60 |
| 333 | 20 | 2 | 90 |
| 333 | 26.6 | 4 | 120 |
| 333 | 40 | 10 | 150 |
| 333 | 66.6 | 20 | 180 |
| 333 | 100 | 33 | 200 |
| 500-1500 | 5-150 | 0.5-50 | 60-300 |
| 1000 | 5 | 1 | 60 |
| 1000 | 20 | 2 | 90 |
| 1000 | 26.6 | 4 | 120 |
| 1000 | 40 | 10 | 150 |
| 1000 | 66.6 | 20 | 180 |
| 1000 | 100 | 33 | 200 |

In some embodiments, one or more of the agents herein are formulated with a pharmaceutically acceptable carrier or adjuvant. The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The agents described herein are preferably administered orally, for example as a component in a pharmaceutical composition. The pharmaceutical compositions may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form.

The pharmaceutical compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

When the agents described herein are administered in combination, both of the agents should be present at dosage levels of between about 1 to 100%, and more preferably between about 5 to 95% of the dosage normally administered in a monotherapy regimen. The agents may be administered separately, as part of a multiple dose regimen. Alternatively, the agents may be part of a single dosage form, mixed together with the compounds of this invention in a single composition.

Preferred unit dosage formulations are compounded for immediate release, though unit dosage formulations compounded for delayed or prolonged release of one or both agents are also disclosed.

In some embodiments, both the biguanide (e.g., metformin) and the meglitinide, e.g., mitiglinide, repaglinide, or nateglinide, preferably mitiglinide, are formulated so that the two agents are simultaneously released, e.g., formulated for immediate or instantaneous release of both agents.

In some embodiments, the agents are formulated in a single unit dose such that the agents are released from the dosage at different times. For example, the meglitinide can be formulated for release in the stomach and the biguanide formulated for release in the small intestine. In some embodiments, the unit dose is formulated to provide a sustained release of one or more of the agents (e.g., the biguanide such as metformin).

In some embodiments, for example where one or more of the agents is administered once per day, the agent is formulated to provide extended release. For example, the agent is formulated with an enteric coating. In an alternative embodiment, the agent, for example, metformin, is formulated using a biphasic controlled release delivery system, thereby providing prolonged gastric residence. For example, in some embodiments, the delivery system includes (1) an inner solid particulate phase formed of substantially uniform granules containing a pharmaceutical having a high water solubility, and one or more hydrophilic polymers, one or more hydrophobic polymers and/or one or more hydrophobic materials such as one or more waxes, fatty alcohols and/or fatty acid esters, and (2) an outer solid continuous phase in which the above granules of inner solid particulate phase are embedded and dispersed throughout, the outer solid continuous phase including one or more hydrophobic polymers, one or more hydrophobic polymers and/or one or more hydrophobic materials such as one or more waxes, fatty alcohols and/or fatty acid esters, which may be compressed into tablets or filled into capsules. In some embodiments, the agent, for example metformin or a meglitinide, is incorporated into polymeric matrices comprised of hydrophilic polymers that swell upon imbibition of water to a size that is large enough to promote retention of the dosage form in the stomach during the fed mode.

In some embodiments, the meglitinide, e.g., repaglinide, mitiglinide, or nateglinide, in the formulation is not formulated with two different release properties, e.g., two different time release properties. For example, the meglitinide is formulated in a single release formulation, a formulation with a single kinetic property. Preferably, the biguanide, e.g., metformin is release with the same kinetics, as the meglitinide.

In some embodiments, the meglitinide, e.g., repaglinide, mitiglinide, or nateglinide, in the formulation is not formulated in a fast-acting and controlled release forms.

In some embodiments, the meglitinide is formulated with a single release properties. For example, it is not present in a modified release form, e.g., a controlled release form.

Compositions disclosed herein can be taken just prior to or with each of three meals, each of two major meals, or one meal. In some embodiments, a composition disclosed herein is administered less number of times in a day than number of meals consumed by a subject, for example, a composition is administer two times daily in a subject who has eaten three or more meals in the day. In other embodiments a composition disclosed herein can be administered once a day or twice a day and need not be administered just before or with a meal.

### EXAMPLES OF SUBJECTS AND DISORDERS

The methods can include administering a biguanide, e.g., an effective amount of metformin, in combination with a meglitinide such as mitiglinide, repaglinide, or nateglinide, e.g., an effective amount of mitiglinide. The methods can be used, e.g., to treat or prevent a metabolic disorder, e.g., metabolic syndrome (e.g., Syndrome X), diabetes, post-prandial hyperglycemia, etc. A “metabolic disorder” refers to a disorder in which one skilled in the art would detect a physiological change in the subject that alters metabolism of at least one substance, e.g., carbohydrates or fats.

Syndrome X

A metabolic syndrome (e.g., Syndrome X and syndrome-associated insulin resistance) is manifested in a patient who presents a group of metabolic risk factors. These factors include: central obesity (excessive fat tissue in and around the abdomen), atherogenic dyslipidemia (blood fat disorders—mainly high triglycerides and low HDL cholesterol—that foster plaque buildups in artery walls); insulin resistance or glucose intolerance (e.g., the body cannot properly use insulin or blood sugar); prothrombotic state (e.g., high fibrinogen or plasminogen activator inhibitor [−1] in the blood); raised blood pressure (130/85 mmHg or higher); and proinflammatory state (e.g., elevated high-sensitivity C-reactive protein in the blood). Overweight/obesity, physical inactivity and genetic factors can contribute to the syndrome. People with a metabolic syndrome are at increased risk of coronary heart disease, other diseases related to plaque buildups in artery walls (e.g., stroke and peripheral vascular disease) and type 2 diabetes. Metabolic syndrome can be closely associated insulin resistance.

Diabetes

In one embodiment, the metabolic disorder is diabetes, e.g., type 2 diabetes mellitus. For example, the patients can be normal (e.g., with respect to blood glucose levels), have impaired glucose tolerance (IGT), so-called pre-diabetic subjects, or diabetic subjects. The patients can have fasting hyperglycemia, e.g., patients that do not otherwise have diabetic characteristics and with fasting glucose levels between 100-125 mg/dL. Examples of diabetes include insulin dependent diabetes mellitus and non-insulin dependent diabetes. For example the method includes administering to a patient having diabetes or at risk of diabetes a combination of compounds described herein. In some instances, a patient can be identified as being at risk of developing diabetes by having impaired glucose tolerance (IGT), or fasting hyperglycemia.

For example, the compounds described herein can be administered to a subject in a therapeutically effective amount to decrease gluconeogenesis, improve glycemic control (e.g., lower fasting blood glucose), or normalize insulin sensitivity. The compounds can be administered to a subject suffering from diabetes or obesity.

Insulin dependent diabetes mellitus (Type 1 diabetes) is an autoimmune disease, where insulitis leads to the destruction of pancreatic J-cells. At the time of clinical onset of type 1 diabetes mellitus, significant number of insulin producing beta cells are destroyed and only 15% to 40% are still capable of insulin production (McCulloch et al. (1991) Diabetes 40:673-679). Beta cell failure results in a life long dependence on daily insulin injections and exposure to the acute and late complication of the disease.

Type 2 diabetes mellitus is a metabolic disease of impaired glucose homeostasis characterized by hyperglycemia, or high blood sugar, as a result of defective insulin action which manifests as insulin resistance, defective insulin secretion, or both. A patient with Type 2 diabetes mellitus has abnormal carbohydrate, lipid, and protein metabolism associated with insulin resistance and/or impaired insulin secretion. The disease leads to pancreatic beta cell destruction and eventually absolute insulin deficiency. Without insulin, high glucose levels remain in the blood. The long term effects of high blood glucose include blindness, renal failure, peripheral neuropathy, inflammatory diseases, glycosylation of neuritic or amyloid plaques which presage Alzheimer's Disease and poor blood circulation to these areas, which can lead to severe ulceration and ultimately foot and ankle amputations. Early detection is critical in preventing patients from reaching this degree of severity. The majority of patients with diabetes have the non-insulin dependent form of diabetes, currently referred to as Type 2 diabetes mellitus.

In some embodiments, a subject can be identified as being normal, prediabetic or diabetic by measuring one or more parameters of the subject's glucose levels. For example, when measuring the post-prandial glucose level of a subject (e.g., measuring the blood glucose of a patient 2 hours after a 100 g glucose drink), a subject defined as normal will have a blood glucose level of <140 mg/dL, a subject defined as prediabetic will have a blood glucose level of 140 to 199 mg/dL, and a subject defined as diabetic will have a blood glucose level of ≧200 mg/dL. When measuring the fasting glucose of a subject, a subject defined as normal will have a fasting blood glucose level of <100 mg/dL, a subject defined as prediabetic will have a fasting blood glucose level of 100-125 mg/dL, and a subject defined as diabetic will have a fasting blood glucose level of >125 mg/dL. A subject can also be defined as prediabetic by having 6-7% glycosylated hemoglobin A1c in the blood, whereas a subject defined as diabetic would have >7% glycosylated hemoglobin A1c in the blood. Methods of the invention can be used to treat or prophylactically treat these subjects.

In some instances, a subject can be treated to lower fasting or post-prandial glucose levels and in other instances a subject can be treated to lower levels of glycosylated hemoglobin A1c or related symptoms.

Hypertension

In another embodiment, the invention includes a method of treating, prophylactically treating, reducing the risk of, or preventing hypertension in a subject using a treatment regime described herein. Hypertension (or high blood pressure) means high pressure (tension) in the arteries (i.e., the vessels that carry blood from the pumping heart to all of the tissues and organs of the body). Normal blood pressure is generally below 120/80; blood pressure between 120/80 and 139/89 is generally considered “pre-hypertension”, and a blood pressure of 140/90 or above is generally considered high blood pressure. The systolic blood pressure, which is the top number, represents the pressure in the arteries as the heart contracts and pumps blood into the arteries. The diastolic pressure, which is the bottom number, represents the pressure in the arteries as the heart relaxes after the contraction. The diastolic pressure, therefore, reflects the minimum pressure to which the arteries are exposed.

An elevation of the systolic and/or diastolic blood pressure increases the risk of developing heart (cardiac) disease, kidney (renal) disease, hardening of the arteries (atherosclerosis or arteriosclerosis), eye damage, and stroke (brain damage). These complications of hypertension are often referred to as end-organ damage because damage to these organs is the end result of chronic (long duration) high blood pressure.

In some embodiments, the method includes administering to the subject an effective amount of meglitinide e.g., mitiglinide, repaglinide, or nateglinide. In some preferred embodiments, the meglitinide is administered in combination with another agent such as a biguanide (e.g., metformin) as in a treatment regime described herein.

In some embodiments, the meglitinide e.g., mitiglinide, repaglinide, or nateglinide is administered in combination with an ARB or ACE inhibitor such as one of the ARB or ACE inhibitors described herein.

Myocardial Infarction

In another embodiment, the invention includes a method of treating, prophylactically treating, reducing the risk of, or preventing (e.g., primary or secondary prevention) a myocardial infarction in a subject using a treatment regime described herein. In some embodiments, the method includes administering to the subject an effective amount of a meglitinide e.g., mitiglinide, repaglinide, or nateglinide. In some preferred embodiments, the meglitinide is administered in combination with another agent such as a biguanide (e.g., metformin). In some preferred embodiments, the method includes secondary prevention of a myocardial infarction, for example, treating a subject who has had a myocardial infarction with mitiglinide to reduce the risk of having an additional myocardial infarction. In some instances the secondary prevention includes combination therapy with mitiglinide an another agent such as metformin.

Combinations with Other Agents:

In some instances the subject can be further treated with a third agent, e.g., an anti-diabetic agent. Examples of anti-diabetic agents include a glucosidase inhibitor, a thiazolidinedione (e.g., TZD), an insulin sensitizer, a glucagon-like peptide-1 (GLP-1), insulin, a PPAR α/γ dual agonist, an aP2 inhibitor and/or a DPP4 inhibitor.

Examples of a glucosidase inhibitor include acarbose (disclosed in U.S. Pat. No. 4,904,769), voglibose, miglitol (disclosed in U.S. Pat. No. 4,639,436), which may be administered in a separate dosage form or the same dosage form.

Examples of a PPARγ agonist includes a thiazolidinedione (e.g., TZD) such as rosiglitazone (AVANDIA®), pioglitazone (ACTOS®), englitazone, and darglitazone, which may be administered in a separate dosage form or the same dosage form.

An example of a DPP4 inhibitor includes PHX1149, which is being developed by Phenomix®.

The compositions described herein can also be administered with an anti-hyperglycemic agent such as insulin or with glucagon-like peptide-1 agonist (GLP-1) or analogs thereof, such as GLP-1(1-36) amide, GLP-1(7-36) amide, GLP-1(7-37) (as disclosed in U.S. Pat. No. 5,614,492, the disclosure of which is incorporated herein by reference), which may be administered via injection, or by transdermal or buccal devices. Treatments can include incretin mimetics such as exenatide (BYETTA®) and DPP-IV inhibitors such as sitagliptin (JANUVIA) and vildagliptin (GALVUS). The compositions described herein can also be administered with an antihypertensive, such as an angiotensin II blocker (ARB) (e.g., irbesartan (AVAPRO), candesartan (ATACAND), losartan (COZAAR), or valsartan (DIOVAN)), or an angiotensin converting enzyme (ACE) inhibitor such as captopril (Capoten), benazepril (Lotensin), enalapril (Vasotec), lisinopril_(Prinivil, Zestril) fosinopril (Monopril), ramipril (Altace), perindopril (Aceon), quinapril (Accupril), moexipril (Univasc), and trandolapril (Mavik).

Where present, the additional agent (e.g., thiazolidenedione, such as troglitazone, rosiglitazone or pioglitazone, the glucosidase inhibitor acarbose, voglibose or miglitol, or insulin) may be employed in formulations as described above and in formulations, amounts and dosing as indicated in the Physicians' Desk Reference.

Titration of a Patient:

Treatment of subjects can be optimized by titrating the subject, for example, such that treatment can be initiated with sub-optimal or no-effect doses of each compound and increased to determine the optimal dose of either a meglitinide such as mitiglinide, repaglinide, or nateglinide or a biguanide such as metformin. Each drug can be co-titrated in a matrix fashion to obtain tailored dosing for the individual.

Treating a subject with a biguanide (e.g., metformin) and/or a meglitinide (e.g., mitiglinide, repaglinide, or nateglinide) can cause side effects. For example, in some instances a patient can have hypoglycemia (e.g., evidenced by lightheadedness and/or dizziness), lactic acidosis, diarrhea, nausea, and/or upset stomach.

The side effects can be modulated to some extent by starting at a low dose and slowly titrating the dose upward, e.g., during the course of treatment, for example over the course of weeks, months or years. Reducing side effects could also improve the efficacy of a combined treatment with a biguanide, preferably metformin and a meglitinide, e.g., mitiglinide, repaglinide, or nateglinide, preferably mitiglinide on an “intention to treat” basis by increasing compliance.

In some embodiments, a patient is titrated to determine the appropriate dosage form of an agent or combination of agents (e.g., combination of metformin with a meglitinide such as mitiglinide, repaglinide, or nateglinide). When used in combination, the agents can be titrated one at a time or can be titrated simultaneously. For example, if a patient is already being treated with a first agent (e.g., metformin), the patient can maintain a constant course, for example, the existing course of treatment for the first agent (e.g., metformin, such as a 1000 mg or 1500 mg daily dose of metformin), while titrating the second agent (e.g., a meglitinide such as mitiglinide, repaglinide, or nateglinide). Alternatively, a patient already being treated with a first agent can be retitrated for that first agent while being titrated for the second agent (e.g., a meglitinide such as mitiglinide, repaglinide, or nateglinide) for example when the patient is initiating a new combination therapy regime.

In some embodiments a patient is being treated with a first agent such as a biguanide and a second agent such as a meglitinide and is changing the treatment regime from a first meglitinide to a second, different meglitinide. Because the patient is not treatment naïve to meglitinides as a class, the patient can, in some embodiments, begin titration of the second, different meglitinide at a relatively higher dose. For example, where a patient has been treated with a first meglitinide such as repaglinide and is being switched to a second meglitinide such as mitiglinide, titration of the second meglitinide (i.e., mitiglinide) can begin at a higher dose than would be generally used for a patient naïve to the class of meglitinides. For example the second meglitinide (i.e., mitiglinide) can begin a titration at a dose of about 10 mgs per day rather than beginning a titration at about 2.5 mgs or 5 mgs per day. Where the second meglitinide is repaglinide, the titration can begin at about 3 mgs per day. Where the second meglitinide is nateglinide, the titration can begin at about 100 mgs per day.

For example, a subject can be instructed to begin a treatment regime having an initial dose of either a biguanide (e.g., metformin) or a meglitinide (e.g., mitiglinide, repaglinide, or nateglinide). A medical professional (e.g., a doctor, nurse or pharmacist) can then assess the subject after the subject has initiated treatment (e.g., 10-14 days after initiation of treatment of 10 mg TID of a meglitinide) to obtain blood glucose readings and assess possible adverse effects. If the subject is tolerating the medication or the combination of medications (e.g., average blood sugar is >140 mg/dl, and there are no unexplainable hypoglycemic episodes <70 mg/dl (hypoglycemia not due to unusual diet or exercise)), the medical professional can increase or decrease the daily dose of the medication accordingly.

For example, a subject can be provided with a starter kit having a range of dosages, e.g., at least two, three, or four different dosages of one or more of a meglitinide, biguanide or combination of the two. Such a kit may contain, for example, a predetermined number of days, e.g., 21 days worth of tablets in total at variant strengths for administration over a consecutive day period to minimize and avoid any adverse effects associated with and to improve tolerance of the medication. More particularly, the starter kit may include a predetermined number, e.g., 7 initial daily doses, 7 increased daily doses and 7 further increased daily doses accompanied with instruction to take one tablet approximately 30 minutes prior to a meal twice daily beginning with the initial dose followed by the increased dose followed by the further increased dose until the tablets are gone.

In some embodiments, a starter kit comprises a plurality of doses of metformin and a meglitinide such as mitiglinide, repaglinide, or nateglinide. In some embodiments, a starter kit of metformin and meglitinide would be given to a subject who has previously received metformin therapy and is now initiating combination therapy of metformin with mitiglinide. In some embodiments the starter kit includes a plurality of doses having a constant dose of metformin, such as 500 mgs, and a variable, increasing dose of mitiglinide, for example, 1, 2.5, 5, 10, 15, 20, 25, 30, 35, or 40 mgs. The starter kit can provide the agents as separate dosage formulations, or alternatively, the starter kit can provide single dosage formulations of the two agents. In embodiments where the starter kit includes combined single unit dosages, exemplary unit dosages are as follows (provided in ratios of metformin:mitiglinide as mg dosages): 500:1, 500:2.5; 500:5, 500:10, 500:15, 500:20, 500:25, 500:30, 500:35, or 500:40.

Other examples of metformin doses include 250 mgs, 750 mgs, and 1000 mgs. Corresponding exemplary starter kits are as follows: 250:1, 250:2.5; 500:5, 250:10, 250:15, 250:20, 250:25, 250:30, 250:35, or 250:40; 750:1, 750:2.5; 750:5, 750:10, 750:15, 750:20, 750:25, 750:30, 750:35, or 750:40; and 1000:1, 1000:2.5; 1000:5, 1000:10, 1000:15, 1000:20, 1000:25, 1000:30, 1000:35, or 1000:40.

Once the starter kit has been completed, the subject could be further titrated by starting the subject on a regimen where the subject increases his daily dose once monthly (e.g., 1500 mg of metformin per day and 15 mg of mitiglinide per day). Once the subject has achieved a dose regime that is effective to improve glycemic control but is without adverse effects, the patient should maintain this course of therapy. The subject can be further monitored (e.g., monitored for levels of blood glucose, such as fasting or post-prandial or hemoglobin 1Ac) to measure the continued effectiveness of the regime. If the subject begins to have decreased glycemic control, then the dose regimen of one or more of a biguanide (e.g., metformin) or meglitinide (e.g., mitiglinide, repaglinide, or nateglinide) can be increased.

It of course should be understood, that after each dosage increase following completion of the starter kit as indicated above, the patient should be evaluated to determine the level of glycemic control and to assess whether the subject is having any adverse effects. In addition, the subject's level of glycemic control should be thoroughly checked before and upon completion of the starter kit, and that should be continually and closely monitored throughout the entire therapeutic treatment.

It therefore should be appreciated by those skilled in this art that the above-regimen represents one example as to how to titrate and treat a patient with decreased glycemic control in accordance with the present invention.

A number of embodiments of the invention have been described. Nevertheless, it will be understood that various modifications may be made without departing from the spirit and scope of the invention. Accordingly, other embodiments are within the scope of the following claim.

What is claimed is:

1. A method of treating or prophylactically treating diabetes in a subject, the method comprising:
administering to a subject a meglitinide, at least twice a day, wherein at least one meal is consumed by the subject without the administration of a dose of a meglitinide, thereby treating said subject and
further comprising administering a biguanide, at least one time per day.

2. The method of claim 1, wherein the meglitinide is mitiglinide.

3. The method of claim 1, wherein the meglitinide is repaglinide.

4. The method of claim 1, wherein the meglitinide is nateglinide.

5. The method of claim 1, wherein the biguanide is metformin.

6. The method of claim 1, wherein the biguanide is administered no more than twice a day.

7. The method of claim 1, wherein a meglitinide is administered with at least one meal but not every meal in a day.

8. The method of claim 7, wherein a first dose is administered with a first meal, and a second does is administered independent of a meal.

9. The method of claim 7, comprising administering a first dose with a first meal; administering a second dose with a second meal; and wherein a third meal is consumed by the subject without a dose of a meglitinide, wherein the terms first, second, and third are used merely to identify the meal and dose and do not imply an order of meal or dose.

10. The method of claim 9, comprising administering a dose with breakfast, or the first meal of the day; administering a dose with the last, or evening meal, and wherein the subject also consumes a third meal or midday meal.

11. The method of claim 7, comprising administering in combination a dose of a meglitinide and a dose of a biguanide with breakfast, or the first meal of the day; administering in combination a dose of a meglitinide and a dose of a biguanide with the last, or evening meal, and wherein the patient also consumes a third meal or midday meal and does not take a dose of a meglitinide with the third or midday meal.

12. The method of claim 11, wherein the meglitinide is mitiglinide.

13. The method of claim 11, wherein the meglitinide is repaglinide.

14. The method of claim 11, wherein the biguanide is metformin.

15. The method of claim 1, wherein no more than two meglitinide administrations per day are administered to the subject.

16. The method of claim 1, wherein no more than two biguanide administrations per day are administered to the subject.

17. The method of claim 16, wherein no more than two meglitinide administrations per day are administered to the subject.

18. The method of claim 1, wherein at least one meglitinide administration is administered with a biguanide administration.

19. The method of claim 18, wherein the meglitinide administration and biguanide administration are a combined unit dosage form.

20. A pharmaceutical composition comprising mitiglinide and a biguanide.

21. The pharmaceutical composition of claim 20, wherein the biguanide is metformin.

22. The pharmaceutical composition of claim 21, comprising a combined salt of metformin:mitiglinide.

23. A method of titrating a subject for a meglitinide, the method comprising:
administering increasing doses of the meglitinide to the subject, and
evaluating the subject for tolerance to the meglitinide as the dose is increased.

24. The method of claim 23, wherein the meglitinide is mitiglinide.

25. The method of claim 23, wherein the meglitinide is repaglinide.

26. The method of claim 23, further comprising administering to the subject a biguanide.

27. The method of claim 26, wherein the biguanide is metformin.

28. The method of claim 23, wherein the patient is not drug naïve for a meglitinide.

29. The method of claim 28, wherein the patient is being switched from a first meglitinide to a second, different meglitinide.

30. The method of claim 29, wherein the second meglitinide is mitiglinide.
